(19) United States
(12) Patent Application Publication
(10) Pub. No.: US 2020/0297206 A1
(43) Pub. Date: Sep. 24, 2020

(54) SYSTEM FOR ASSESSING A HEALTH CONDITION OF A USER

(71) Applicant: VIVIOR AG, Zuerich (CH)

(72) Inventors: Pavel Zakharov, Volketswil (CH); Michael Mrochen, Eglisau (CH)

(21) Appl. No.: 16/498,054

(22) PCT Filed: Mar. 29, 2018
(86) PCT No.: PCT/EP2018/058135

§ 371 Date: Sep. 26, 2019

(30) Foreign Application Priority Data
Mar. 29, 2017 (EP) 17163664.0

(51) Int. Cl. A61B 3/113; A61B 3/10; A61B 3/00; A61B 5/00; G16H 50/70; G16H 50/20

(52) U.S. Cl. CPC A61B 3/113; A61B 3/101; A61B 3/0008; A61B 2560/04; G16H 50/70; G16H 50/20; A61B 5/6803

(57) ABSTRACT

The invention relates to a system for assessing a health condition of a user comprises a sensor unit, a monitoring unit and a storage unit. The sensor unit comprises at least one eye sensor. The at least one eye sensor is adapted to obtain an optical signal reflected from an eye and/or surrounding tissues of the user. The sensor unit can be mounted on a wearable device. The monitoring unit is connected to the sensor unit. The monitoring unit is adapted to derive data related to an eye activity of the user by processing the optical signal. The data related to the eye activity of the user is included in the optical signal. The storage unit is connected to the monitoring unit. The storage unit is adapted to store the derived data related to the eye activity of the user and recorded data. The monitoring unit is further adapted to obtain the recorded data from the storage unit. The monitoring unit is further adapted to assess the health condition of the user by comparing the recorded data with the derived data related to the eye activity of the user. Further, the invention relates to a method for assessing the health condition of the user.

The present invention relates to a system for assessing a health condition of a user and a method for assessing the health condition of the user.

Usually, systems for eye blink detection are applied in the case of sleep warning of drivers. Thereby, a path of light from a source to a detector may be interrupted by a blinking eye and can thus be identified. Further, reflectance based eye tracking systems can detect a viewing direction of a user by triggering an event when the user is looking in a direction of a light source or a light sensor. If the user is looking straight ahead, an incident ray from the light source is substantially reflected from the scattering sclera to give a first level outputted by the light sensor. Next, with the eye turning to look in the direction of the light source, the ray principally strikes the iris or pupil, which produces a reduction in the light level outputted by the light sensor. This may cause an electrical output of the light sensor to change significantly and to change a control device such as a relay switch to which it is connected for effecting any desired control activity. Further, a blinking detection technique may be based on a pair of a light source and a detector. Further, reflection intensities between an eyelid and an eyeball can be effectively utilized to provide a device which is not subject to accidental activation due to minor eye movement. For example, with the proper arrangement of light source and detector when the eye is open, most of an incident light will be absorbed by the eyeball, and only a small portion is reflected. When the eye is closed, a greater portion of the incident light is reflected by the eyelids as compared to the eyeball.

It is an object of the present invention to improve a treatment of a patient suffering, for example, from a dry eye phenomenon and medically support the patient.

According to a first aspect, a system for assessing a health condition of a user comprises a sensor unit, a monitoring unit and a storage unit. The sensor unit comprises at least one eye sensor. The at least one eye sensor is adapted to obtain an optical signal reflected from an eye of the user. The sensor unit can be mounted on a wearable device. The monitoring unit is connected to the sensor unit. The monitoring unit is adapted to derive data related to an eye activity of the user by processing the optical signal. The data related to the eye activity of the user is included in the optical signal. The storage unit is connected to the monitoring unit. The storage unit is adapted to store the derived data related to the eye activity of the user and recorded data. The monitoring unit is further adapted to obtain the recorded data from the storage unit. The monitoring unit is further adapted to assess the health condition of the user by comparing the recorded data with the derived data related to the eye activity of the user.

The sensor unit and the monitoring unit may be connected via a radio channel (e.g. Bluetooth, ANT+, WiFi), optical channel (e.g. LiFi, infrared channel) or digital bus (e.g. USB, I2C, SPI).

The advantage of the system lies in that it provides a tool for supporting a patient with information related to the patient's health condition. The sensor unit enables the patient to acquire data gathering for different users and for himself/herself. This generates a datapool making a patient's decision on a medical behaviour more precise.

The term “eye activity” may be understood as an adjustment of the eye physiological state. This includes blinks, eye movements, such as rotational movement, pupil size changes, accommodation, tear film quality, tear film motion, etc. Further eye activity may be referred to by a parameter of interest. The parameter of interest may be a frequency of blinks (i.e. blinks/minute), interblink interval (i.e. seconds), eye movements (i.e. vertical vs. horizontal), pupil radius, blink completeness and other such as refraction properties.

The wearable device may be adapted to be wearable on the head of the user. The sensor unit may be mounted on the wearable device. The wearable device may be a spectacles frame. This can be a dedicated frame or normal glasses (both prescribed and non-prescribed) suitable to hold a sensor attachment.

The monitoring unit may be a separate device or can also be mounted on the wearable device. The monitoring unit may be further adapted to carry out the processing of (raw) data, evaluating parameters of the eye activity, and controlling an exchange of data between the sensor and the monitoring unit, the sensor and the storage unit and/or the monitoring unit and the storage unit. The monitoring unit may be in the form of a wearable or mobile device, such as a smartphone, tablet, desktop, laptop computer or dashboard software. The monitoring unit may be located on a frame of a wearable device or can be a separate unit. The monitoring unit may comprise a controller adapted to display information to the user via a screen or indicator. The controller may be adapted to accept an input from the user via buttons, gestures (both head, hands' and arms' movements) or via other means. The controller may have additional sensors adapted to derive a blinking pattern of the user as a reference, such as a camera of the smartphone or a web-camera on the computer. The monitoring unit can be further adapted to access a historical calibration database comprising data from the user or other users. The monitoring unit can further carry out calibration processes. The monitoring unit can be adapted to synchronize the derived/historical/calibration data with a central database. The monitoring unit may be further adapted to be dependent on a user's input complying with the derived data to be shared. When the derived data is to be shared, other users can be able to use the derived data for their own use.

The storage unit may be in the form of a cloud, internet server, mobile phone storage etc.

The at least one eye sensor may be an optical sensor which itself can be or includes a light detector. The at least one eye sensor may be referred to as at least one eye activity sensor. The at least one eye sensor may be arranged on glasses of the wearable device, such that a light reflected from a light source can be optimally received. The at least one eye sensor may be in the form of a single point photodetector (i.e. Photodiode or phototransistor), multiple spatially separated point detector (i.e. row of photodiodes) and/or detector array (i.e. point detectors arranged in a grid or a camera). The camera can be of a CCD or CMOS type.

The recorded data can comprise stored data related to the eye activity of the user. The recorded data can further comprise stored data related to the eye activity of other users. The recorded data can further comprise stored data related to the health condition of the user. The recorded data can further comprise stored data related to an input of the user. The recorded data can further comprise stored data related to a health condition or an input of the other users.

The recorded data can be previously stored data. The recorded data can be historical data. The recorded data can indicate the health condition of the user. The recorded data can indicate a health condition of another user or other users.

The recorded data provide the advantage of enabling a better medical treatment of a patient being provided with such a system.

The optical signal can originate from a light source.

The light source can be an ambient light. The light source can be an artificial light source. The artificial light source can be mountable on the wearable device.

The system can further comprise at least one light source. The at least one light source can be adapted to transmit the optical signal to an eye and/or surrounding tissues of the user. The at least one light source can be arranged to transmit the optical signal to an eye and/or surrounding tissues of the user. The at least one light source can further be calibrated. The at least one light source can further be mountable on the wearable device to transmit the optical signal to the eye and/or surrounding tissues of the user. The at least one eye sensor can further be calibrated to be in alignment with the light source for the monitoring unit to optimally derive data related to the eye activity of the user.

The system can further comprise at least one light detector. The at least one light detector can be adapted to receive the optical signal from an eye and/or surrounding tissues of the user. The at least one light detector can be arranged to receive the optical signal from an eye of the user. The at least one light detector can further be calibrated. The at least one light detector can further be mountable on the wearable device to receive the optical signal reflected from the eye structures, eyelid and/or other eye surrounding tissues of the user. The at least one light detector can further be calibrated to be in alignment with the at least one light source for the monitoring unit to optimally derive data related to the eye activity of the user based on the received optical signal.

A combination of a light source/ambient light with a light detector has the advantage of sensitize the deriving of the data.

The light source can be a single light source, multiple light sources and/or ambient light. The light technology underlying the light source may be a light emitting diode (LED), a superluminiscent diode (SLD), a laser diode and/or specifically directed waveguides to define a path of light. The light source may be further arranged on glasses of the wearable device, such that they are optimally aligned for improving processing results. The light source may be further arranged externally from the wearable device, such as on a desk, computer screen or a mobile device (i.e. smartphone or table).

The eye sensor can be able to be calibrated to a personal condition of the user. The personal condition can comprise an eye size of the user. The personal condition can comprise a relative position of the frame to a position of the eyes of the user. The personal condition can comprise a relative position of the wearable device to the position of the eyes of the user.

A calibration can have the advantage of making the system adaptable to a special user and making the derived data more precise and comparable to other user's extracted data, such as the recorded data.

The monitoring unit may be further adapted to relate (raw) optical signals to an eye activity of the user using the calibration data. The optical signals may be reflections from eye structures, an eyelid and/or other surrounding tissues of the user.

The term “calibration data” may be understood as a data used by an algorithm to relate measured raw signals, such as the derived signal, to the actual eye activity. Calibration data can be in the form of raw measurement data, reference data or context data. The sensor unit and the monitoring unit are adapted to calibrate raw signals (raw signals may comprise context, reference and optical data) by obtaining information about a blink state from an independent reference signal. Such a reference signal can be a direct user input provided through the glasses of the wearable device, like pressing a button on the frame of the wearable device, tapping on the frame detected by an accelerometer on the frame, a head or hand gesture. This feature can be further implemented in a smartphone or mobile device app. For example, the reference signal for the blink detection can be blink detection vision algorithm analysing images from the smartphone camera imaging user's eyes.

The sensor unit may further comprise a context sensor. The context sensor can be adapted to detect another signal related to an environment of the user. The context sensor can be arranged in the sensor unit. The context sensor can be arranged in the monitoring unit. The monitoring unit can further be adapted to derive environmental data included in the other signal by processing the other signal. The storage unit can further be adapted to store the derived environmental data. The monitoring unit can further be adapted to assess the health condition of the user by comparing the recorded data with the derived data related to the eye activity of the user and the derived environmental data. The context sensor may be arranged on the sensor unit and/or the monitoring unit. The context sensor may be a physiological sensor or ambient/environmental sensor. The sensor unit may further comprise a motion sensor, such as an accelerometer, a magnetometer and/or a gyroscope, an environmental sensor and/or an additional physiological sensor, or a plurality thereof. The accelerometer/magnetometer/gyroscope may be adapted to detect an orientation of the head of the user. The accelerometer/magnetometer/gyroscope may be adapted to detect user activity, such as walking, running, reading, talking, etc. The accelerometer/magnetometer/gyroscope may be adapted to detect a user input, such as tapping, shaking, gestures, etc. The physiological sensor may be adapted to measure vital signs, such as a heart rate, blood oxygenation and/or electro dermal activity, etc. The ambient/environmental sensor may be in the form of a proximity sensor, for example on a frame of the wearable device. The proximity sensor may be directed in a direction of sight and may detect distances to objects. The ambient/environmental sensor may be in the form of and ambient light sensor adapted to measure intensity and/or spectral content of visible and/or infrared and/or ultraviolet ranges, wherein the monitoring unit is further adapted to calculate a UV index to relate it to the eye activity. The ambient/environmental sensor may be in the form of a temperature sensor adapted to measure a temperature, wherein the monitoring unit is adapted to relate the temperature to an activity of the user. The ambient/environmental sensor may be in the form of a humidity sensor adapted to measure a humidity, wherein the monitoring unit is adapted to relate the humidity to an activity of the user. The ambient/environmental sensor may be in the form of a pressure sensor adapted to measure a pressure, wherein the monitoring unit is adapted to relate the pressure to an activity of the user. The ambient/environmental sensor may be in the form of an environmental pollution monitoring unit adapted to determine an amount of pollution in the air, wherein the monitoring unit is further adapted to derive data related to the eye activity, such as blinking triggers based on the pollution. The ambient/environmental sensor may be in the form of a location based sensor adapted to determine the user's location in non-shadowed environments, wherein the monitoring unit is further adapted to calculate a position based on the communication system or an internet connection. The ambient/environmental sensor may be in the form of a camera. The camera may be adapted to e.g. periodically capture images of an environment of the user. The camera can be triggered by the user. The monitoring unit can be further adapted to derive data related to the context comprising information about the environment.

An advantage of the context sensor is to build in environmental and ambient influences in the data to be derived and the stored/recorded data which makes it more flexible in processing, such that further medical issues can be observed and treated. It further makes meaningful physiological interpretation possible.

The term “environmental data” may be understood as context data, which is information about the user. Context data can be information not directly derived from the eye, such as heart rate, perspiration, head orientation, age, etc. Context data can be information about the user's environment, such as temperature, humidity or position. Context data can further be information from a user, provided via text, voice and/or image input, about information, such as being itchy, tired and/or sleepy.

The system can further comprise a user interface. The user interface can be adapted to receive an input from the user. The user can indicate whether the health condition corresponds to the derived eye activity data and/or the derived environmental data. The indication can be performed by an input of the user via the input interface.

The user interface can have the advantage of weighting a determined result of the health condition, such that a health condition can be weighted less when it is determined to be false and it can be weighted less when it is determined right. This as a result can then be stored as recorded data with a specified weighting according to the user's input.

The recorded data can further comprise previously stored calibration data and previously stored environmental data from the user and/or other users.

Further, the monitoring unit and the sensor unit can be adapted to receive calibration data by a calibration process performed by the user. The calibration data and the previously stored calibration data can be data which is used by an algorithm to convert a raw signal from a detector, such as the light detector or the sensor unit, and context data to a physiological parameter of interest. At least part of the calibration data and previously stored calibration data may be in a form of parameters for the algorithm for such a conversion. Further, at least part of the calibration data or previously stored calibration data may be in a form of computer instructions for such a conversion.

The user interface can be further adapted to indicate whether a physical and/or psychological abnormality occurred based on the comparing.

The monitoring unit can further comprise an alarm unit. The alarm unit can be adapted to indicate to a user that a physical and/or psychological abnormality occurred based on the comparing. The indication may be in the form of images, for example projected on the glasses of the wearable device or a vibration alarm or just visible light. Further the indication can be dependent on physiological status of the user, such as informing the user to blink, to relax the eyes and/or to reduce an exposure to harmful environment, and/or dependent on a status of the monitoring unit or sensor unit, such as a battery status, connection status etc. The indication can further be dependent on external conditions or events, such as an incoming phone call, drop of outside temperature etc. The alarm unit may further be adapted to be in the form of an input unit and to receive a user input via a button/accelerometer based tap detector. The monitor unit may further be adapted based on an indication from the alarm unit to display information to the user and/or alarm the user.

The alarm unit can have the advantage of providing a user/patient with direct information, such that the user/patient can take quick responsibility in a medical or treatment scenario where fast reaction is required.

The system can further comprise an additional sensor, for example a smartphone camera. A front and/or back camera of the smartphone can be used. The additional sensor is adapted to obtain another optical signal reflected from the eye of the user. The monitoring unit, for example the smartphone, can be adapted to perform an image analysis algorithm. The image analysis algorithm is adapted to identify blinks of the user and to relate the identified blinks of the user to the data related to the eye activity.

The system can further comprise an eye movement sensor adapted to sense movements of eyes (and/or pupils) of the user and/or a distance measurement sensor adapted to measure distances to one or more objects from the distance measurement sensor in at least one direction. The monitoring unit may be further adapted to measure a direction of gaze of the user using the sensed movements, weight the measured distances based on a deviation of the at least one direction from the direction of the gaze, and calculate a viewing distance between the one or more objects and the user based on the weighted distances.

The system can be further adapted to monitor eye movements in order to derive the direction of gaze. The eye movement sensor may sense movements of the eyes. The monitoring unit may derive the gaze direction of the eyes. The eye movement sensor may be equipped in the sensor unit of the system or the function of the eye movement sensor may be performed in the sensor unit. The movements of the eyes may correspond to movements of the eyes (and/or pupils). The monitoring unit may derive the gaze direction based on the movements of the eyes or pupils.

The system can further comprise a distance measurement sensor which measures distances (or viewing distances) to one or more objects from the distance measurement sensor at least in one direction surrounding the user. The monitoring unit may derive the object or scene that the user is specifically looking at. The object or scene may correspond to an environment or activity of the user using the measured distances. For example, typical distances in a certain direction for an activity of reading a book can be defined and stored in the storage unit. In case the typical distances match the measured distances (with or without offset), the monitoring unit may determine that the user is reading a book or the object corresponds to the book.

If the distance measurement sensor measures the distances in a single direction, weighting the measured distances based on an alignment of the distance measurement sensor and the direction of gaze may be applied. For example, more value (larger weight) may be given to the measured distances, when the derived direction of gaze is codirectional with the distance measurement sensor, i.e. eyes are looking in the direction of the distance measurement sensor. In the opposite case, when the direction of gaze significantly deviates from the sensing direction of the distance measurement sensor, distance measurements can be devalued (assigned smaller weight) or even be discarded from the statistics. If the distance measurement sensor is capable of sampling in multiple directions simultaneously as in the case of a camera or sequentially as in the case of a scanner, or both, the system may derive distances from the measurements in directions aligned with the direction of gaze.

The eye movement sensor or the sensor unit may further sense at least one of coordinated movements of eyes (and/or pupils), size of the pupils or change of the lens shape of the user. The monitoring unit may further determine an accommodation effort using at least one of a vergence derived from the sensed movements of the eyes, the size of the pupils and the change of the lens shape.

The monitoring unit may calculate the viewing distance of the user based on the determined accommodation effort. The viewing distance can be defined as a distance to a point where the user is looking at.

The eye activity may be also understood as the accommodation effort. The system can be further adapted to monitor the accommodation effort of the eye. When human eyes focus on an object, they perform coordinated adjustments in vergence, shape of the lens to change optical power and, correspondingly, focal length and pupil size. For example, monitoring of positions of both eyes can allow detection of the vergence, which is a simultaneous movement of both eyes in the opposite direction. Eyes move towards each other while focusing on near objects and move away of each over while focusing on distant objects. Changes of the shape of the lens can be monitored by tracking the reflections of the probing light from surfaces of the lens (for example, by analysing Purkinje reflections, such as P3 and P4). When focusing on a near object, pupils constrict in order to minimize image blurring. Pupil size can be measured with imaging or any other suitable method. The system can detect the accommodation by detection of pupil size changes. During the detection of the accommodation from the pupil size, the system may compensate effects to the size of the pupil due to brightness which may be measured with the context sensors, such as an ambient light sensor.

By tracking the accommodation effort using any of the mentioned features or a combination of two of more of them: vergence, lens shape change, pupil size, the system can track viewing distances that a user is using. Statistics of the viewing distances (which is related to visual lifestyle of the user) can be utilized to advise/warn the user in real time to adjust visual behaviour of the user to more healthy one, for example, if the user is consistently focusing on near objects for a prolonged time, like during reading or working on a computer, the system can advise to take a break, relax eyes by looking on distance objects, etc. Analysis of the visual behaviour can be performed for the more extended periods and feedback can be given in a more general behavioural way, like “you should spend less time in front of the computer” or “spend more time outdoors”.

The measured or derived eye activity/visual behaviour statistics can be used by a health care practitioner to customize treatment for a patient. For example, an unusual blinking pattern can indicate dry eye disease and thus prevent the surgeon from performing refractive surgery until the condition improves. The measured or derived eye activity/visual behaviour statistics can be used by the user to optimise the performance of the vision and/or reduce eye stress so that the user turns to a healthier lifestyle. The user can also improve the environment, like increasing humidity in the working area to reduce dry eye related symptoms, and/or adjust the computer monitor position to reduce load on the neck, etc.

According to a second aspect, a method for assessing a health condition of a user comprises obtaining, by a sensor unit, an optical signal reflected from an eye of the user. The sensor unit can be mounted on a wearable device. The method further comprises deriving, by a monitoring unit connected to the sensor unit, data related to an eye activity of the user by processing the optical signal. The data related to the eye activity of the user is included in the optical signal. The method further comprises storing, by a storage unit connected to the monitoring unit, the derived data related to the eye activity of the user and recorded data. The method further comprises obtaining, by the monitoring unit, the recorded data from the storage unit. The method further comprises assessing, by the monitoring unit, the health condition of the user by comparing the recorded data with the derived data related to the eye activity of the user.

Even if some of the aspects described above have been described in reference to the system, these aspects may also apply to the method. Likewise, the aspects described above in relation to the method may be applicable in a corresponding manner to the system.

Other objects, features, advantages and applications will become apparent from the following description of non-limiting embodiments with reference to the accompanying drawings. In the drawings, all described and/or illustrated features, alone or in any combination form the subject matter disclosed therein, irrespective of their grouping in the claims or their relations/references. The dimensions and proportions of components or parts shown in the figures are not necessarily to scale; these dimensions and proportions may differ from illustrations in the figures and implemented embodiments.

The figures show:

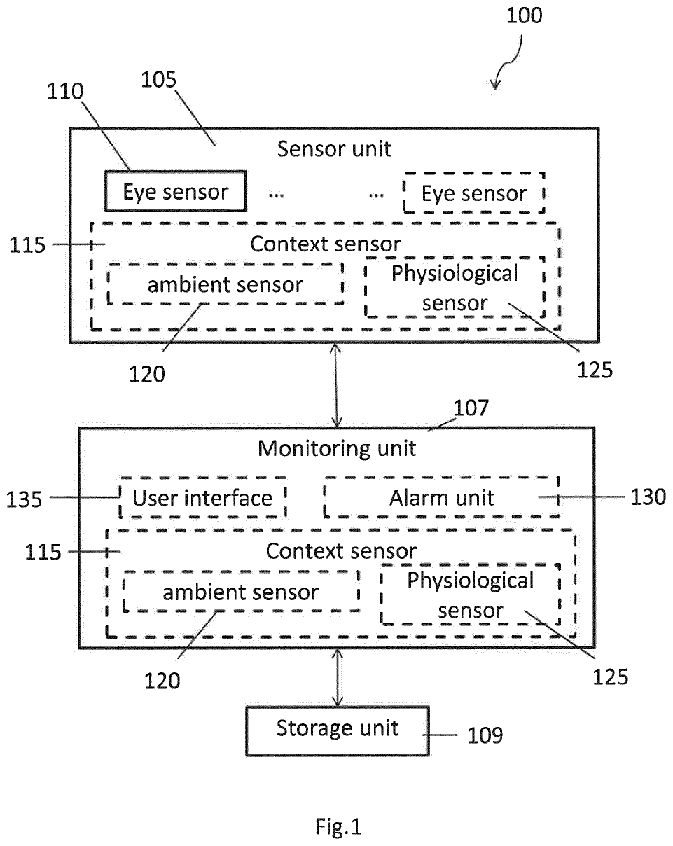

FIG. 1 schematically illustrates a system implementation according to an embodiment;

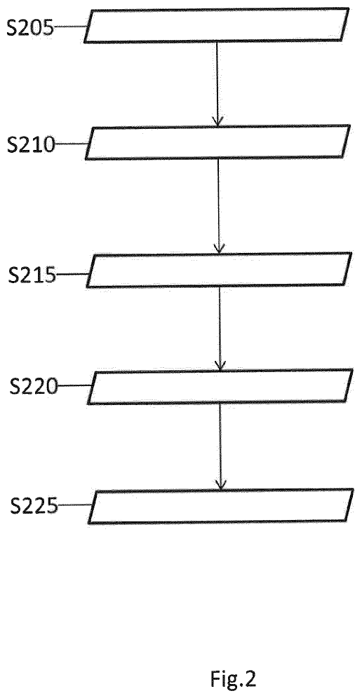

FIG. 2 schematically illustrates a method implementation according to an embodiment;

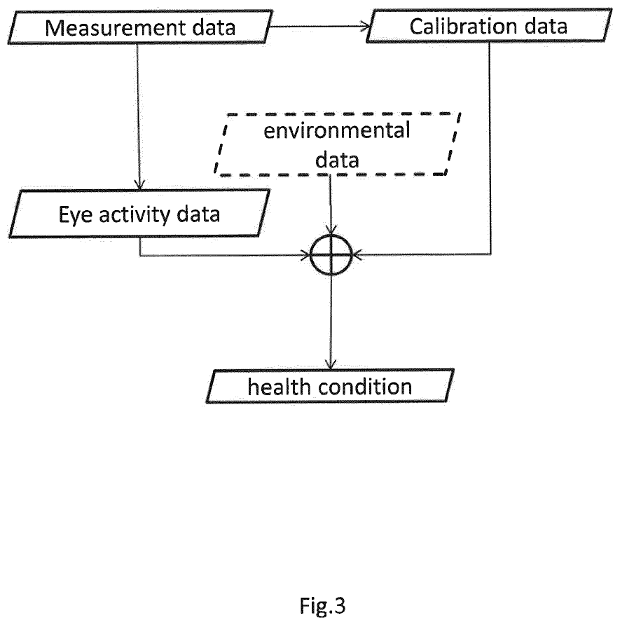

FIG. 3 schematically illustrates a flow chart according to a method implementation of an embodiment;

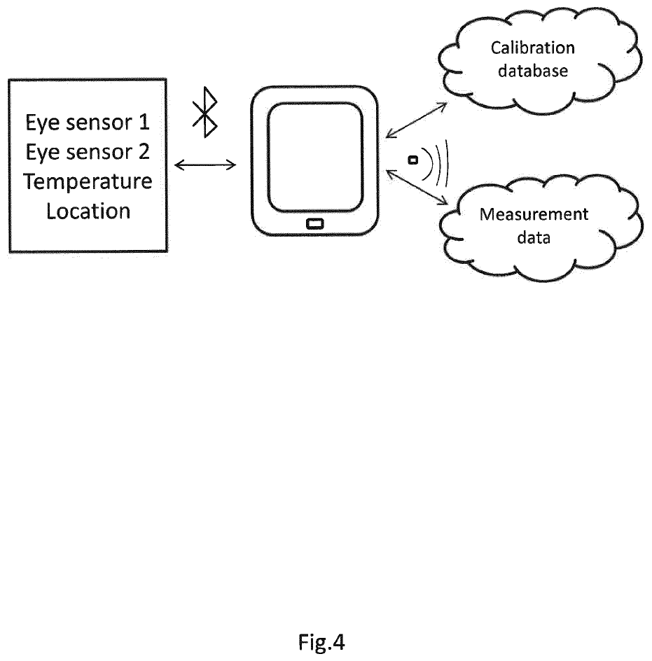

FIG. 4 schematically illustrates an exemplary system implementation according to an embodiment;

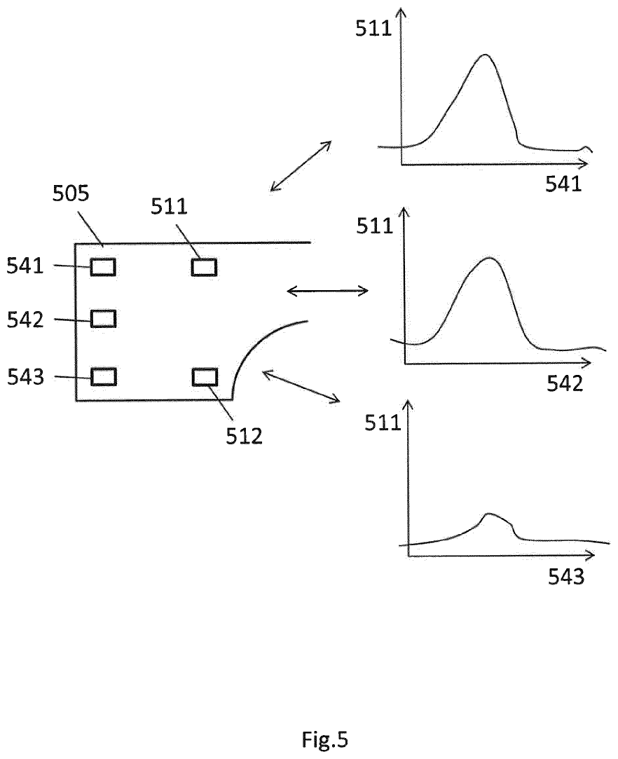

FIG. 5 schematically illustrates a light detector and light source implementation on a wearable device according to an embodiment;

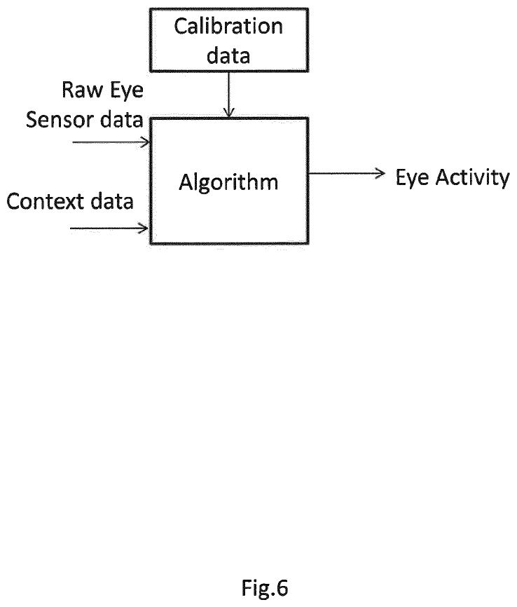

FIG. 6 schematically illustrates a flow diagram exemplifying an algorithmic process according to an embodiment; and

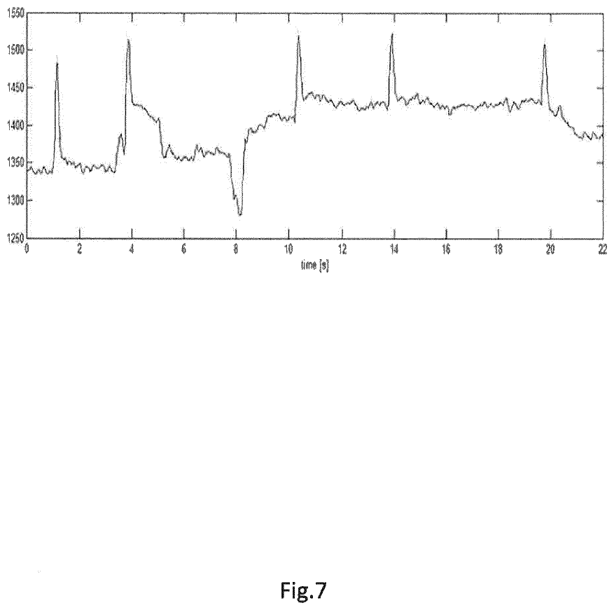

FIG. 7 schematically illustrates a diagram representing a blinking signal comprised in an optical signal according to an embodiment.

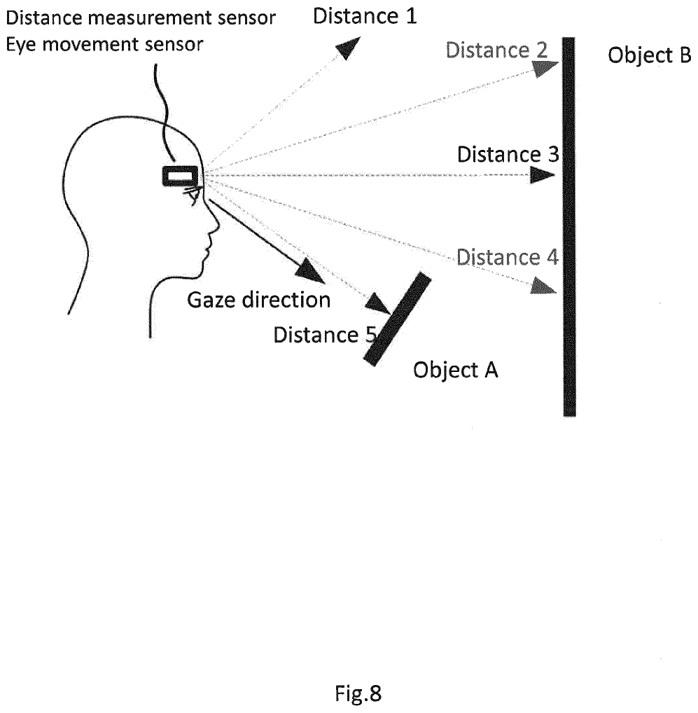

FIG. 8 schematically illustrates an example vertically for weighting measured distances with regard to a gaze direction of a user.

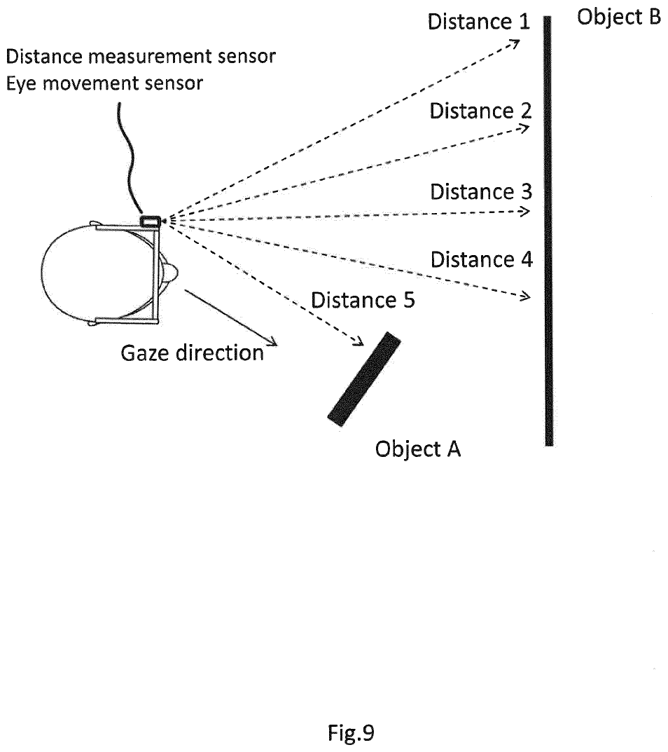

FIG. 9 schematically illustrates an example horizontally for weighting measured distances with regard to a gaze direction of a user.

The variants of the functional and operational aspects as well as their functional and operational aspects described herein are only for a better understanding of its structure, its functions and properties; they do not limit the disclosure to the embodiments. The figures are partially schematic, said essential properties and effects are clearly shown enlarged in part in order to clarify the functions, active principles, embodiments and technical characteristics. Every operation, every principle, every technical aspect and every feature that/which is disclosed in the figures or in the text is/are able to be combined with all claims, each feature in the text and the other figures, other modes of operation, principles, technical refinements and features that are included in this disclosure, or result from it, so that all possible combinations are assigned to the devices and methods described. They also include combinations of all individual comments in the text, that is, in each section of the description, in the claims and combinations between different variations in the text, in the claims and in the figures, and can be made to subject-matter of further claims. The claims do not limit the disclosure and therefore the possible combinations of all identified characteristics among themselves. All features disclosed are explicitly also individually and in combination with all other features disclosed herein.

FIG. 1 schematically illustrates a system 100 implementation according to an embodiment of the invention. The system 100 for assessing a health condition of a user comprises a sensor unit 105, a monitoring unit 107 and a storage unit 109. The sensor unit 105 comprises at least one eye sensor 110. The at least one eye sensor 110 is adapted to obtain an optical signal reflected from an eye of the user. The optical signal can be generated by an artificial light or can be gathered by the at least one eye sensor 110 using an ambient light reflected by at least one of the user's eyes. The artificial light can be connected to the sensor unit 105 or the monitoring unit 107. The sensor unit 105 can be mounted on a wearable device. The wearable device can be adapted to mount the sensor unit 105, the monitoring unit 107, the storage unit 109 and/or the artificial light/light source. The monitoring unit 107 is connected to the sensor unit 105. This connection can be established via a Bluetooth connection. The monitoring unit 107 is adapted to derive data related to an eye activity of the user by processing the optical signal. The data related to the eye activity of the user is included in the optical signal. The storage unit 109 is connected to the monitoring unit 107. This connection may be established via an internet connection. The storage unit 109 is adapted to store the derived data related to the eye activity of the user and recorded data. The monitoring unit 107 is further adapted to obtain the recorded data from the storage unit 109. The monitoring unit 107 is further adapted to assess the health condition of the user by comparing the recorded data with the derived data related to the eye activity of the user.

Data related to the eye activity of the user can be data related to blinking and eye movements, for example to assess dry eye monitoring. It is an advantage of the present invention to be able to extract parameters of interest from the data related to the eye activity. For example, the storage unit 109 can be a database of calibration data available from other users. Further this calibration data can be recorded data from the user who is currently wearing the wearable device. Further, it is advantages to use context data, such as environmental and physiology data to further improve an accuracy of the data related to the eye activity. Further, the context data can enhance the data for more meaningful physiologic interpretation. In FIG. 1, the context data is collected by the context sensor 115. The context sensor 115 can comprise an ambient sensor 120 and/or a physiological sensor 125. The ambient sensor 120 can be adapted and arranged to collect data from the surrounding environment. The physiological sensor 125 can be adapted and arranged to collect data related to human vitals of the user. Further, the sensor unit and the monitoring unit can be separate units or comprised in the same unit. The monitoring unit 107 can comprise a user interface 135, an alarm unit 130 and the context sensor 115. The user interface 135 can be adapted and arranged to receive an user input from the user wearing the wearable device, such as the spectacles mounting the sensor unit. Further, the user interface 135 can be adapted to interact with the monitoring unit, such that a user is able to weigh an importance of gathered/collected data related to the eye activity. The alarm unit 130 may be adapted and arranged to alarm/signal to the user that something related to the eye activity or the environment or his/her physiology may need to be adjusted. The monitoring unit 107 can be connected to the storage unit 109, in such a way that the monitoring unit 107 can derive data, which might be recorded data/stored data or currently processed data from the storage unit 109 in order to compare it with the currently processed/derived data. The storage unit 109 can be a server/database, cloud or any kind of storage which is able to be accessed over the Internet.

As for example, one can consider blinking activity and a physiological dry eye condition as a medical problem. The eye sensor mountable on a frame of a pair of spectacles gathers eye activity data providing after a calibration a signal of blinking events. For example, a user exhibits specific blinking statistics such as frequency in the simplest case. Then a product specific application (app) on a smartphone can automatically query a cloud database and takes into account other parameters, such as user's activity or ambient humidity, which may also be monitored by another sensor such as a context sensor 115 mounted on the wearable device. The results could indicate that a majority of users with such a blinking pattern and under those specific ambient conditions later reported all were diagnosed with an onset of the dry eye problem. The system can be used so that the user can be advised to take specific actions. Predictive analytics can be performed on the cloud based on historical data to warn the user in advance. In an even simpler case, an algorithm can predict that a long period of non-blinking through the day might result in itchiness and redness of the eyes in the evening, for example while watching TV or working on the PC which might be self-reported or detected by reference sensors such as a smartphone camera. It is an advantage of the present invention to use historical data and relate to blinking to the user's health condition. This is also achieved by the concept of connecting the device to access the cloud data analytics which allows to solve the problem of a device calibration. The device can be understood as the sensor unit 105. Further, automatic calibration of the device with reference states such as eyes closed or open, looking to the left or right, up or down, etc. based on a reference method for example camera of the smartphone to which the device is connected. The simple use case scenario is that the user puts the wearable device on, for example glasses, starts a control application on the monitoring unit, which activates the camera of the smartphone for monitoring an eye activity. Then the user is to open/close the eyes which is automatically detected by the application capturing images from the smartphone camera and related to the signals measured by the sensor unit/eye sensor mounted on the wearable device. After calibration has completed, the smartphone application deactivates the camera and the user can proceed with his or her normal daily activities, while the sensor unit 105 in connection with the monitoring unit 107 is enabled to detect a blinking pattern of the user. Further, the calibration of raw signals gathered by the sensor unit 105 is based on the historical/recorded data available for the same user of other users in combination with the context data such as age, sex, facial features, etc. The system 100 enables the user to infer physiological and/or psychological information based on the historical/recorded data from the same or other users and related to the self-reported status. The physiological data can be an onset of dry eye disease. The psychological data can be stress, drowsiness, etc.

Eye activity can be understood as an adjustment of the eyes' psychological state. This includes blinks, eye movements including rotation, pupil size changes, accommodation, etc. Context data can be information about the user, which is not directly derived from the eye (heart rate, perspiration, head orientation, age, etc.), the user's environment (temperature, humidity, location) and text fields, voice input or images to add information from the user himself/herself (i.e. itchy, tired, sleepy). Calibration data can be the data which is used by an algorithm to convert raw signals from the sensor unit and context data to the physiological parameters of interest. At least part of the calibration data may be in a form of parameters for the algorithm for such conversions. At least part of the calibration data may be in a form of computer instructions for such conversions. The sensor unit 105 and the monitoring unit 107 may be able to detect and relate raw sensor measurement signals like light reflections from the eye to the actual eye activity using calibration data. The derived eye activity parameters of interest might be a frequency of blinks, such as blinks per minute, interblink interval in seconds, eye movements in vertical and horizontal direction, pupil radius, blink completeness and other such as refraction. The purpose of the sensor unit 105 is to obtain raw signals from the eye and the relevant context data. The sensor unit 105 may be mounted on a spectacles frame. This spectacles frame can be a dedicated frame or normal glasses suitable to hold the sensor unit 105. The monitoring unit 107 can be a separate device also mounted on the spectacles frame. The monitoring unit 107 can carry out the processing of the raw data, evaluating the parameters and controlling and exchanging of data between the sensor unit 105 and the storage unit 109, which can be a network storage (cloud).

The sensor unit 105 may be implemented in a glasses frame with an attached proximity sensor and an infrared LED located in front of one or both of the user's eyes. The proximity sensor may be connected to a microcontroller board (which may also be attached on the glasses' frame such as the spectacles temple) via a digital bus. The proximity sensor can also act as an ambient light sensor. The sensor modulates the intensity of LED and subtracts ambient background. It delivers a signal related to the proximity of an object in front of the user. Closer (or more reflective) objects lead to a higher signal level. The microcontroller board may have a battery as a power source, micro USB interface for charging and a 3-D accelerometer and temperature sensor. The board can communicate with the monitoring unit 107, such as a smartphone or tablet, via a low-energy Bluetooth interface. The smartphone/tablet may run an app which reads out the measurements of the proximity sensor, accelerometer and temperature sensor and may further be adapted to store them into a data file for post-processing. The smartphone/tablet may perform an analysis of the signal and to identify blinks by predefined signatures of a signal change. This is then shown as statistics of the blinks to the user. Further, the at least one eye sensor 110 may contain multiple sources and/or detectors in order to be able to identify and use optimal combinations/mixtures of signals for an individual or for a current fitting of the glasses/spectacles.

To understand the principle of the present invention, FIG. 2 schematically illustrates a method implementation according to an embodiment.

FIG. 2 schematically shows the method according to an embodiment of the invention for assessing a health condition of a user. The method comprises obtaining S205, by a sensor unit, an optical signal reflected from an eye of the user. The sensor unit can be mounted on a wearable device. The method further comprises deriving S210, by a monitoring unit connected to the sensor unit, data related to an eye activity of the user by processing the optical signal. The data related to the eye activity of the user is included in the optical signal. The method further comprises storing S215, by a storage unit connected to the monitoring unit, the derived data related to the eye activity of the user and recorded data. The method further comprises obtaining S220, by the monitoring unit, the recorded data from the storage unit. The method further comprises assessing S225, by the monitoring unit, the health condition of the user by comparing the recorded data with the derived data related to the eye activity of the user.

For a better understanding, another embodiment of the method for assessing a health condition of a user is shown in FIG. 3.

FIG. 3 schematically illustrates a flow chart according to a method implementation of an embodiment of the invention. In FIG. 3, measurement data, calibration data, eye activity data and environmental data are illustrated to be combined in order to extract a health condition. The measurement data can be data related to an eye activity currently measured by the sensor unit/eye sensor according to FIG. 1. The measurement data can be calibration data obtained by a calibration technique performed by the user before using the wearable device comprising the sensor unit. Further, the calibration data can be data from the same or other users obtained in earlier sessions of using the wearable device. The wearable device can be a frame/spectacles. In order to obtain calibration data, the user/other users need to wear the wearable device in order to gather calibration data/historical data. The historical data, herein also referred to as recorded/stored data, can be used in a step of comparison with the currently derived data while the user uses the wearable device to obtain data. Further, environmental data can be gathered in conjunction with the calibration data and the eye activity data in order to more accurately derive and/or assess a health condition of the user.

In order to ease the understanding of the method implementation according to the foregoing disclosure, an exemplary system implementation is shown in FIG. 4.

FIG. 4 schematically illustrates an exemplary system implementation according to an embodiment of the invention. Different kinds of sensors embodied as the sensor unit according to FIG. 1 are illustrated on the left-hand side of the figure, such as eye sensor 1 and the eye sensor 2, and a temperature and location sensor. This is just an exemplary sensor arrangement, such that it can also only comprise one eye sensor and one of the temperature and the location sensors for example. The sensor unit is for example connected to the monitoring unit, here illustrated as a smartphone device, via a Bluetooth connection. The monitoring unit itself is for example connected to a calibration database and a measurement database via wireless communication. For example, the bluetooth connection and the wireless communication connection can be switched on and off via a user interface by the user. This enables the monitoring unit to extract data from the calibration database in combination with the measurement data. The measurement data can be the derived data, and the calibration data from the calibration database can be the recorded data. The monitoring unit 107 may comprise another type of sensor, such as a location-based sensor for example GPS, GLONASS or GNSS sensors. In some situations in which it is difficult to know a user's location, the user may aid in the determination of his or her location and or context information via an input. The user input can be processed by the user interface, for example comprised in the monitoring unit. If the location sensor provides data making it difficult to determine if a user is for example in a car or a bus, the monitoring unit and a cloud server communicating with the monitoring unit may present a query to the user asking if he or she took the bus or the car. Similar queries may occur for locations other than vehicular contexts. For example, if the data related to the eye activity, the user's psychological or physiological state and/or the environment indicates that the user completed a specific task, such as a vigorous workout, but there is no location data that indicates that the user went to a gym, the user may be asked if he/she were to the gym today.

In order to illustrate how data is derived by a wearable device according to an embodiment of the invention, FIG. 5 illustrates an exemplary detector/source arrangement with corresponding diagrams comprising resulting data.

FIG. 5 schematically illustrates a light detector/sensor and light source implementation on a wearable device according to an embodiment of the invention. The wearable device 505 can comprise light sources 541, 542 and 543. The wearable device can further comprise light detectors 511 and 512. On the right-hand side of FIG. 5, diagrams for the relation between the three different light sources 541, 542 and 543 with the light detector 511 are illustrated. The shortest distance between the light source and the light detector happens to lead to the highest amount of output data, thereby leading to a higher peak in the related diagram. The lowest amount of output data happens to correspond to the longest distance between the light source and the light detector, as shown in FIG. 5. The eyes of different users can be located in different positions in relation to the frame of the wearable device 505, light source and light detector. For example, for a user with eyes located closer to the top of the frame of the wearable device 505, the light detector may receive a better signal from a combination of the light source 541 and the light detector 511. For a user with eyes closer to the bottom of the frame, a combination of the light source 543 and the light detector 512 may be preferred.

In order to illustrate how the resulting data can be used in obtaining an eye activity, FIG. 6 schematically illustrates the use of an algorithm for obtaining an eye activity by combining context data and raw eye sensor data, which is also referred to as the optical signal in this disclosure. The raw eye sensor data can be the detected data by the eye sensor.

FIG. 6 schematically illustrates a flow diagram exemplifying an algorithmic process according to an embodiment of the invention. Raw eye sensor data and context data can be gathered and used as input data for the algorithm. Further, calibration data can be used as another input data for the algorithm. Calibration data may be understood as data used by the algorithm to relate the measured raw signals to the actual eye activity. This may be performed by a mapping algorithm. The calibration data may consist of raw measurement data, reference data and context data. The system may be able to calibrate the signal by obtaining information about the blink state from an independent reference signal. Such reference signal may be a direct user input provided through the user interface. The user interface may be arranged on the wearable device. The user input may be represented by pressing a button on the frame of the wearable device, tapping on the frame (for example single tap means eyes open, double tap means eyes closed) detected by an accelerometer being arranged on the frame of the wearable device or a head gesture (for example nodding indicates blink, shaking head indicates eyes open). The user input can be given on a controller with means of pressing a button, tapping if the controller hardware/software allows this kind of operation. This may also be implemented in an app by clicking, or shaking for example the smartphone. When the algorithm is performed, data related to the eye activity is extracted by a combination of the data, such as it is done in FIG. 3.

In order to illustrate the data related to an eye activity, FIG. 7 illustrates a timely variation of the data gathered by an eye sensor.

FIG. 7 schematically illustrates a diagram representing a blinking signal comprised in an optical signal according to an embodiment of the invention. This data is extracted from a sensor output showing an illustration of a blinking signal as detected from an eye reflection.

FIG. 8 schematically illustrates an example vertically for weighting measured distances with regard to a gaze direction of a user, and FIG. 9 schematically illustrates an example horizontally for weighting measured distances with regard to a gaze direction of a user. The distance measurement sensor may sense distances 1, 2, 3, 4 to object B and distance 5 to object A. The eye movement sensor may measure the direction of gaze as indicated in the figure. In this example, the deviation between distance 1 and the gaze direction is the largest, and the deviations of distances 2, 3, 4, 5 from the gaze direction reduce in the order from 2 to 5. Thus, the largest weight may be given to distance 5 (closest to the gaze direction) when the system measures the distances such that the distance of distance 5 can be regarded as the most important measurement for deriving the viewing distance of the user, whereas the distance of distance 1 (furthest away from the gaze direction) can be taken into account as the least important measurement. Distances 2 to 4 may be weighted accordingly, wherein distance 4 may be considered more important than distance 3 and distance 3 may be considered more important than distance 2.

In order to obtain a reference signal, the system according to an embodiment of the present invention can comprise an additional sensor. For example the additional sensor can be comprised in the monitoring unit. For example, if the monitoring unit is a smartphone, the front/back camera can be used and an image analysis algorithm can be adapted to identify blinks of the user and relate them to a primary blink signal without a direct user input via the user interface. This may be referred to as passive monitoring, wherein the reference data analysis can be performed in real-time and/or retrospectively. The retrospective analysis can benefit from a direct user input in a manual or semi-automatic mode. For example in the manual mode, the user is shown the images and is asked to judge the status of the eye (for example blink or open eye). In the semi-automatic mode, the algorithm is identifying the eye status and only shows the user the result with the reference data and asks to confirm or reject the result (for example left or right swipe on the smartphone screen to accept or reject results respectively). As the calibration data can comprise context data, the calibration data can be related to additional physiological or ambient data to improve the accuracy of the eye activity detection. Context data can have a form of static information, like age, body weight, skin type, eyes colour, information about health, etc., an image of the face or in a form of monitored parameters like a user's movement, ambient temperature, etc. Context data can also be used in relation with the eye activity data in order to derive context related statistics.

1-17. (canceled)

18. A system for assessing a health condition of a user comprising:
a sensor unit comprising at least one eye sensor adapted to obtain an optical signal reflected from an eye of the user, wherein the sensor unit can be mounted on a wearable device;
a monitoring unit connected to the sensor unit and adapted to derive data related to an eye activity of the user by processing the optical signal, wherein the data related to the eye activity of the user is included in the optical signal;
a storage unit connected to the monitoring unit and adapted to store the derived data related to the eye activity of the user and recorded data; and
wherein the monitoring unit is further adapted to obtain the recorded data from the storage unit and to assess the health condition of the user by comparing the recorded data with the derived data related to the eye activity of the user.

19. The system according to claim 18, wherein the recorded data comprises stored data related to the eye activity of the user, stored data related to the eye activity of other users, stored data related to the health condition of the user and/or stored data related to a health condition of the other users.

20. The system according to claim 18, wherein the recorded data is previously stored data and/or historical data, which indicates the health condition of the user and/or a health condition of another user or other users.

21. The system according to claim 18, wherein the optical signal originates from a light source, wherein the light source is an ambient light and/or an artificial light source, the artificial light source being mountable on the wearable device.

22. The system according to claim 18, further comprising:
at least one light source adapted and arranged to transmit the optical signal to an eye and/or surrounding tissues of the user, wherein the at least one light source can further be calibrated and is mountable on the wearable device to transmit the optical signal to the eye and/or surrounding tissues of the user; and
wherein the at least one eye sensor can further be calibrated to be in alignment with the light source for the monitoring unit to optimally derive data related to the eye activity of the user.

23. The system according to claim 22, further comprising:
at least one light detector adapted and arranged to receive the optical signal from an eye and/or surrounding tissues of the user, wherein the at least one light detector can further be calibrated and is mountable on the wearable device to receive the optical signal from the eye and/or surrounding tissues of the user; and
wherein the at least one light detector can further be calibrated to be in alignment with the at least one light source for the monitoring unit to optimally derive data related to the eye activity of the user based on the received optical signal.

24. The system according to claim 18, wherein the eye sensor is able to be calibrated to a personal condition of the user comprising an eye size of the user and/or a relative position of the frame or the wearable device to a position of the eyes of the user.

25. The system according to claim 18, wherein the sensor unit further comprises:
a context sensor adapted to detect another signal related to an environment of the user, wherein the context sensor is arranged in the sensor unit or the monitoring unit;
wherein the monitoring unit is further adapted to derive environmental data included in the other signal by processing the other signal;
wherein the storage unit is further adapted to store the derived environmental data; and
wherein the monitoring unit is further adapted to assess the health condition of the user by comparing the recorded data with the derived data related to the eye activity of the user and the derived environmental data.

26. The system according to claim 18, further comprising:
a user interface adapted to receive an input from the user, wherein the user indicates whether the health condition corresponds to the derived eye activity data and/or the derived environmental data.

27. The system according to claim 26, wherein the user interface is further adapted to indicate whether a physical and/or psychological abnormality occurred based on the comparing.

28. The system according to claim 18, wherein the recorded data comprises previously stored calibration data and previously stored environmental data from the user and/or other users.

29. The system according to claim 18, wherein the monitoring unit further comprises:
an alarm unit adapted to indicate to a user that a physical and/or psychological abnormality occurred based on the comparing.

30. The system according to claim 18, further comprising:
an additional sensor adapted to obtain another optical signal reflected from the eye of the user; and
wherein the monitoring unit is adapted to perform an image analysis algorithm, which is adapted to identify blinks of the user and relate the identified blinks of the user to the data related to the eye activity.

31. The system according to claim 18, further comprising:
an eye movement sensor adapted to sense movements of the eyes of the user; and
a distance measurement sensor adapted to measure distances to one or more objects from the distance measurement sensor in at least one direction,
wherein the monitoring unit is further adapted to measure a direction of gaze of the user using the sensed movements, weight the measured distances based on a deviation of the at least one direction from the direction of the gaze, and calculate a viewing distance between the one or more objects and the user based on the weighted distances.

32. The system according to claim 18,
wherein the eye movement sensor or the sensor unit is further adapted to sense at least one of movements of the eyes, size of the pupils or change of a lens shape of the user,
wherein the monitoring unit is further adapted to determine an accommodation effort using at least one of a vergence derived from the sensed movements of the eyes, the size of the pupils and the change of the lens shape.

33. The system according to claim 32, wherein the monitoring unit is further adapted to calculate the viewing distance of the user based on the determined accommodation effort.

34. A method for assessing a health condition of a user comprising:
obtaining, by a sensor unit, an optical signal reflected from an eye of the user, wherein the sensor unit can be mounted on a wearable device;
deriving, by a monitoring unit connected to the sensor unit, data related to an eye activity of the user by processing the optical signal, wherein the data related to the eye activity of the user is included in the optical signal;
storing, by a storage unit connected to the monitoring unit, the derived data related to the eye activity of the user and recorded data;
obtaining, by the monitoring unit, the recorded data from the storage unit; and
assessing, by the monitoring unit the health condition of the user by comparing the recorded data with the derived data related to the eye activity of the user.
